(19) United States
(12) Patent Application Publication
(10) Pub. No.: US 2014/0093980 A1
(43) Pub. Date: Apr. 3, 2014

(54) DISSOLVABLE BRIDGES FOR MANIPULATING FLUID VOLUMES AND ASSOCIATED DEVICES, SYSTEMS AND METHODS

(71) Applicant: University of Washington through its Center for Commercialization, Seattle, WA (US)

(72) Inventors: Elain S. Fu, Corvallis, OR (US); Barry Lutz, Seattle, WA (US); Jared Houghtaling, Edmonds, WA (US); Tinny Liang, Sammamish, WA (US)

(21) Appl. No.: 14/043,664

(22) Filed: Oct. 1, 2013

Related U.S. Application Data
(60) Provisional application No. 61/708,227, filed on Oct. 1, 2012.

(51) Int. Cl. G01N 33/558

(52) U.S. Cl. CPC G01N 33/558
USPC 436/501; 422/69

(57) ABSTRACT

The present technology is directed to capillarity-based devices for performing chemical processes and associated system and methods. In one embodiment, for example, a device can include a source configured to receive one or more fluids, a first material adjacent to and in fluid connection with the source, a second material, and a dissolvable volume-metering element positioned between the first material and the second material. The volume-metering element can be configured to provide a fluid connection between the first material and the second material. The volume-metering element can also be configured to at least partially dissolve and break the fluid connection between the first material and second material once a predetermined volume of fluid flows therethrough.

### CROSS-REFERENCE TO RELATED APPLICATION

This application claims priority to U.S. Provisional Patent Application No. 61/708,227, filed Oct. 1, 2012, and incorporated herein by reference in its entirety.

### TECHNICAL FIELD

The present technology is generally related to capillarity-based devices for performing chemical processes and associated systems and methods. In particular, several embodiments are directed toward a capillarity-based device that makes use of a volume-metering element between adjacent porous membranes to perform microfluidic analyses.

### BACKGROUND

Porous membranes are often used in conventional lateral flow and flow-through cartridges, in which flow of fluid occurs by wicking through the membrane (either laterally or transversely) onto an absorbent pad Immunoassays take advantage of porous wick systems to measure and analyze analyte samples. The dependence on wicking to generate flow greatly limits control over assay conditions. Specifically, lateral flow assays are often limited to a single step in which sample (and buffer) is added to the sample pad, and the sample flows by capillary action (i.e., wicking) along the pad. Capillarity provides the force needed to provide a nearly continuous flow of fluid from one point to another, causing reagents stored in dry form to be transported along the device and to pass over regions that contain immobilized capture molecules. These devices are typically restricted to simple one-shot detection chemistries like colored nanoparticles that do not provide the sensitivity possible with multistep-detection chemistries, such as enzymatic amplification. They are also rarely quantitative.

Microfluidic systems that include open fluid channels for the flow of buffers, samples, and reagents can inherently be made much more sophisticated, and it is possible to use them to carry out a very large number of fluid-processing steps. Such microfluidic systems usually incorporate a complex disposable, which leads to unavoidably high per-test manufacturing costs and the need for expensive external pumps and valves to move fluids. While microfluidic devices can inherently be very flexible in the functions that they perform, they are also inherently complicated and expensive. Additionally, the devices that have been made that support complex function are usually quite complex themselves. For example, some polymeric laminate cartridges currently developed contain as many as 23 different layers, each of which must be separately manufactured and bonded to the others.

### BRIEF DESCRIPTION OF THE DRAWINGS

FIGS. 1A-1C are a series of time-lapsed side views of a pathway having a volume-metering element configured in accordance with an embodiment of the technology.

FIG. 2 is a perspective view of a volume-metering element configured in accordance with an embodiment of the present technology.

FIGS. 3 and 4 are graphs illustrating volumes of fluid delivered for various volume-metering elements configured in accordance with embodiments of the technology.

FIG. 5A is a top view of a capillarity-based device in the open position configured in accordance with an embodiment of the technology.

FIG. 5B is a series of time-lapsed top views of the capillarity-based device of FIG. 5A in the closed position and after fluid has been added configured in accordance with an embodiment of the technology.

FIGS. 6A-6D show a series of time-lapsed top views of a capillarity-based device having multiple pathways configured in accordance with an embodiment of the present technology.

FIG. 7 is a top view of a capillarity-based device in the open position configured in accordance with an embodiment of the present technology.

### DETAILED DESCRIPTION

The present technology describes various embodiments of devices for processing, analyzing, detecting, measuring and separating fluids. The devices can be used to perform these processes on a microfluidic scale, and with control over fluid and reagent transport. In one embodiment, for example, a device for performing chemical processes can include a source configured to contain one or more fluids, a first material adjacent to and in fluid connection with the source, a second material, and a dissolvable volume-metering element positioned between the first material and the second material. The volume-metering element can be configured to provide a fluid connection between the first material and the second material and dissolve once a predetermined volume of fluid flows therethrough.

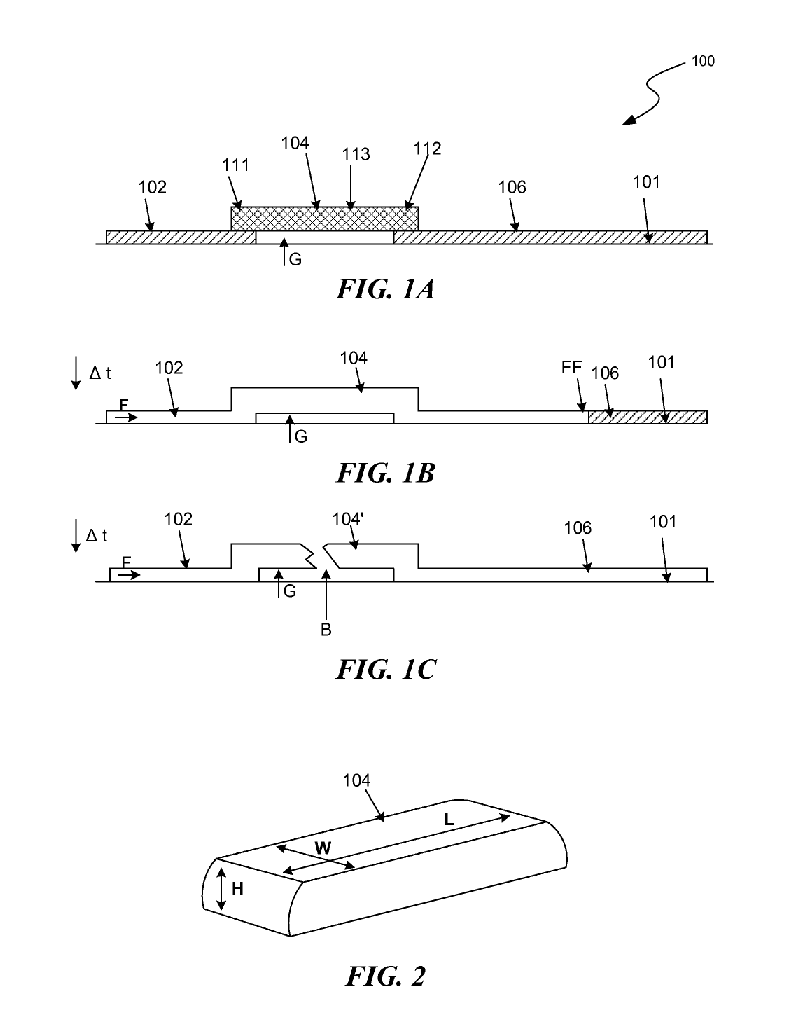

Specific details of several embodiments of the technology are described below with reference to FIGS. 1A-7. Other details describing well-known structures and systems often associated with capillarity-based devices, biomedical diagnostics, immunoassays, etc. have not been set forth in the following disclosure to avoid unnecessarily obscuring the description of the various embodiments of the technology. Many of the details, dimensions, angles, and other features shown in the Figures are merely illustrative of particular embodiments of the technology. Accordingly, other embodiments can have other details, dimensions, angles, and features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 1A-7.

FIGS. 1A-1C area series of time-lapsed side views of a pathway 100 on a supporting surface 101 configured in accordance with an embodiment of the technology. As shown in FIG. 1A, the pathway 100 includes a first porous material or feeder material 102 and a second porous material or delivery material 106 connected by a volume-metering element 104 therebetween. The volume-metering element 104 can have a first portion 111 in contact with the first porous material 102, a second portion 112 in contact with the second porous material 106, and a third portion 113 between the first portion 111 and the second portion 112 and separated from the supporting surface 101 by a gap G. The volume-metering element 104 can comprise a dissolvable or soluble material configured to automatically and independently control or modify a volume of fluid flow between the first material 102 and the second material 106. The first and second materials 102, 106 can include, for example, porous materials such as paper, glass fiber, polyester, nitrocellulose, cellulose, polymer membranes (e.g., cellulose acetate, cellulose esters, polysulfone, polyether sulfone, polyacrilonitrile, polyamide, polyimide, polyethylene and polypropylene, polytetrafluoroethylene, polyvinylidene fluoride, polyvinylchloride, etc.) and other suitable materials. In other embodiments, however, the first material 102 and/or second material 106 may include different materials and/or have a different arrangement.

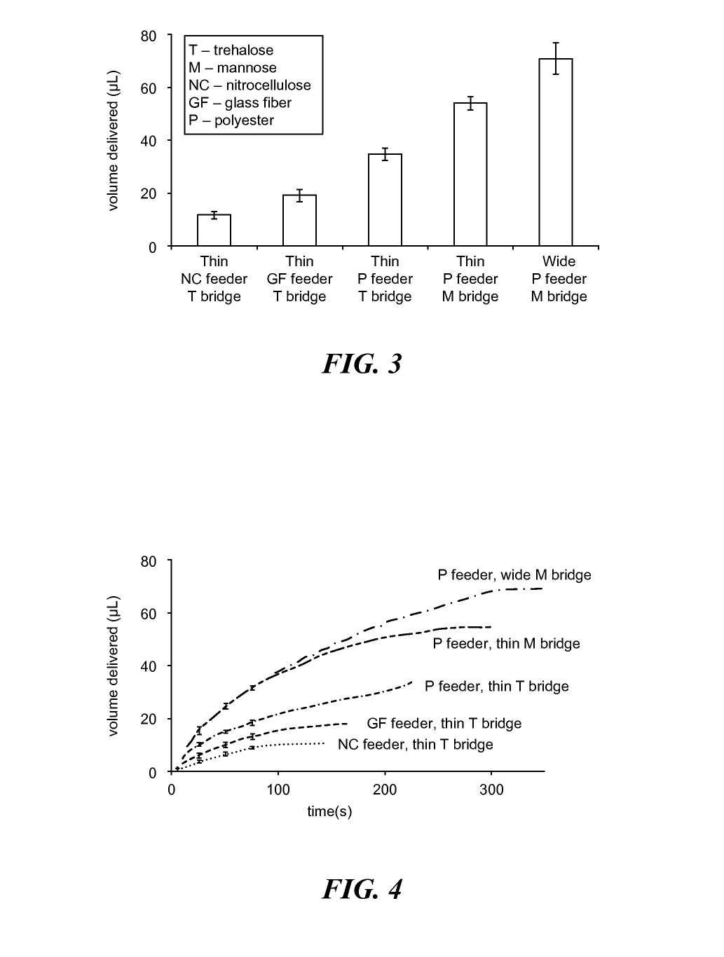

Referring next to FIG. 1B, when fluid F is added to the first material 102, the fluid F wicks (e.g., by capillarity force) from the first material 102 to the volume-metering element 104 to the second material 106. As such, the volume-metering element 104 initially functions as a bridge connecting the first material 102 and the second material 106. Over time, the fluid F dissolves the soluble material comprising the volume-metering element 104 so that once a precise volume of fluid F passes through the element 104, the element 104 or at least a portion of the element 104 breaks B (as shown in FIG. 1C) and permanently disconnects the first material 102 from the second material 106. As discussed in greater detail below with reference to FIGS. 2-4, the delivered volume can be precisely tailored by adjusting one or more pathway parameters, such as the cross-sectional area of the volume-metering element 104, the material composition of the volume-metering element 104, the flow rate of fluid into the element 104 from the first material, the choice of first material 102, and/or the choice of second material 106.

FIG. 2 is a perspective view of the volume-metering element 104 configured in accordance with the present technology. As shown in FIG. 2, the volume-metering element 104 can have a generally rectangular shape. In some embodiments, the volume-metering element 104 may be made of one or more sugars. For example, the volume-metering element can comprise one or more monosaccharides (e.g., mannose, dextrose, fructose, galactose, etc), disaccharides (e.g., trehalose, sucrose, lactose, maltose, etc.), sugar alcohols (e.g,. mannitol, xylitol, sorbitol, etc.), polysaccharides (e.g., dextrans, maltodextrin, starch, etc.), one or more dextran derivatives (dextran sulfate) and/or other suitable sugars. Sugars are compatible with most common lateral flow assay reagents and, as a result, are often used in such assays for preservation of a dried gold label conjugated to antibody. In some embodiments, for example, volume-metering elements 104 composed of high concentrations of sugar solutions can be used with the generation of a downstream lateral flow assay signal in a lateral flow format. In particular embodiments, for example, the volume-metering element 104 can include partially or fully saturated solutions of trehalose spiked with antigen and applied to a lateral flow strip containing dried gold label conjugated to antibody and patterned with PfHRP2 capture antibody downstream. In other embodiments, however, the volume-metering element 104 can have any suitable shape (e.g., square, circle, oval, octagon, ellipse, etc.), size, or configuration, and can be made of any solvent soluble composition capable of bridging a physical gap between two or more porous matrices and predictably dissolving and separating as described in the present disclosure. For example, in some embodiments the volume-metering element 104 can comprise a salt, alginate, and/or carrageenan.

The volume-metering element 104 can have a material composition, length L, width W, height H and/or cross-sectional area designed to pass a pre-defined volume of fluid before dissolving and breaking the fluid connectivity of the pathway 100. For example, in some embodiments the volume-metering element 104 can have a length L between about 6.5 and 8.5 mm (e.g., about 7.5 mm), a width W between about 2 mm and about 4 mm (e.g., about 3 mm), and a height H between about 0.1 mm and about 1.0 mm (e.g., about 0.2 mm, 0.3, 0.4 mm, 0.5 mm, 0.6 mm, etc.). The approximate volume of fluid passed by the volume-metering element 104 can be measured using the location of the fluid front FF (FIG. 1B) within the second material 106 at the time of element 104 disconnection or shut-off. The volume delivered, V, at each time point, t, can be approximated by the equation V(t)=(P*h*A*t), where P is the porosity of the second material 106, h is the height of the second material, and A is the area occupied by the fluid F parallel to flow.

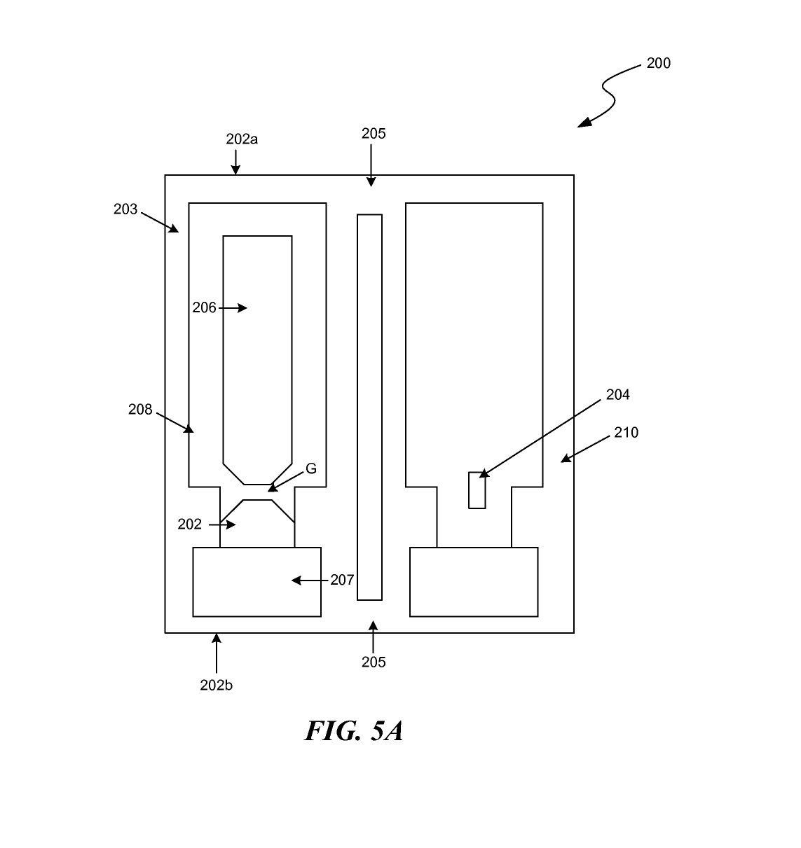

As previously mentioned, the passable volume allowed by the volume-metering element 104 can be tailored by adjusting one or more pathway parameters. FIG. 3, for example, is a graph displaying the volume of fluid delivered for each volume-metering element 104 where the first material 102, the cross-sectional area of the volume-metering element 104, and the material composition of the volume-metering element 104 were adjusted to achieve a range of volumes delivered (e.g., between about 10 μl and about 80 μl. As shown in FIG. 3 and without being bound by theory, it is generally believed that (1) a volume-metering element 104 having a greater width W will pass a greater volume than a volume-metering element having a smaller width W, (2) the material composition of the volume-metering element 104 can affect the passable volume (e.g., all else generally equal, mannose can pass a larger volume than trehalose), and (3) the material composition of the first or feeder material can affect the passable volume (e.g., all else generally equal, glass passes a great volume than polyester that passes a greater volume than nitrocellulose). FIG. 4 is a graph displaying the change in volume of fluid delivered over time for each of the combinations of pathway parameters shown in FIG. 3. As shown, the volume of fluid exiting the volume-metering element 104 is affected by the flow-rate of fluid entering the element 104 from the first or feeder material. It will be appreciated that the specific examples illustrated in FIGS. 3 and 4 are merely representative of particular embodiments of the present technology, and that the first material 102, the second material 106, and/or volume-metering element 104 may have different arrangements and/or different features in other embodiments.

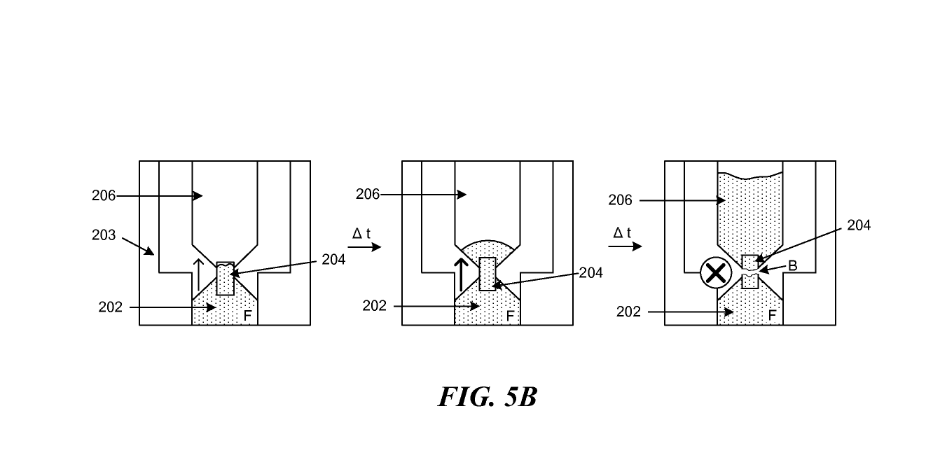

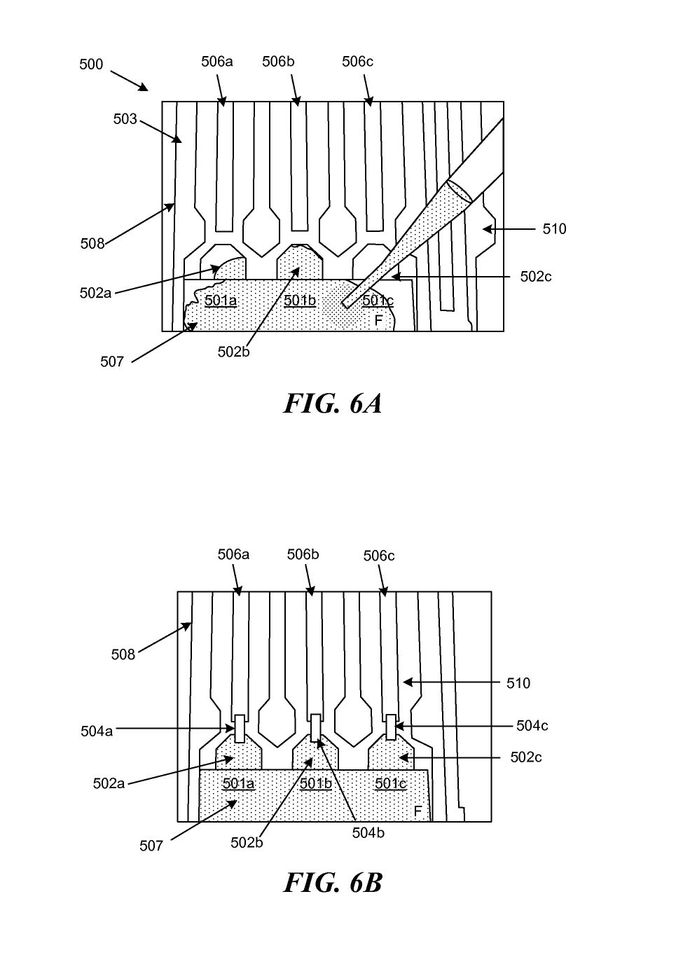

FIG. 5A is a top view of a microfluidic device or analyzer 200 configured in accordance with an embodiment of the present technology. The device 200 can include a thin, foldable housing 202 moveable between an open position (FIG. 5A) and a closed position (a portion of which is shown in FIG. 5B). The housing 203 can have a first end 202a opposite a second end 202b, and a bottom layer 208 and a top layer 210 connected by one or more flexible connectors or hinges 205. The bottom layer 208 of the housing 203 can support at least a portion of one or more fluid pathways 201 and the top layer 210 can have a volume-metering element 204 removably attached thereto. It should be noted that although various aspects of the device 200 are described as “top” or “bottom,” such descriptors are for illustrative purposes only and do not limit the device 200 or any component thereof to a specific orientation.

The fluid pathway 201 can include a first material 202 separated from a second material 206 by a gap G. The first and second materials 202 and 206 may be generally similar to the first and second materials 102 and 106 described above, or they may have a different configuration. In some embodiments, the pathway 201 can optionally include a fluid source 207 adjacent to the first material 202 proximate the first end 202a of the housing 203. The fluid source 207 can be configured to receive and contain a volume of fluid F (e.g., from a pipette) and supply at least a portion of that volume to the first material 202 during the assay. In other embodiments, the device 200 does not include a source 207 and fluid is delivered directly to the first material 202. The volume-metering element 204 can be positioned on the top layer 210 so that when the top layer 210 is folded onto the bottom layer 208 (or vice versa), the volume-metering element 204 aligns with the gap G between the first material 202 and the second material 206, thereby providing a bridge between the first and second materials 202, 206.

In operation, fluid F is loaded into the source 207 and the housing is moved into the closed position to bring the volume-metering element 204 into contact with the first material 202, thereby completing the pathway 201. Within the pathway 201, fluid flows by capillarity force from the source 207 to the first material 202, to the volume-metering element 204, and finally to the second material 206. FIG. 5B, for example, is a series of time-lapsed top views of the pathway 201 after fluid F has been added to the source 207 and the housing 203 is placed in the closed position. As shown, once a precise volume of fluid F flows through the volume-metering element 204, the element 204 dissolves to the point of breaking (break B shown in FIG. 5B), thereby permanently disconnecting fluid F flow between the first material 202 and the second material 206.

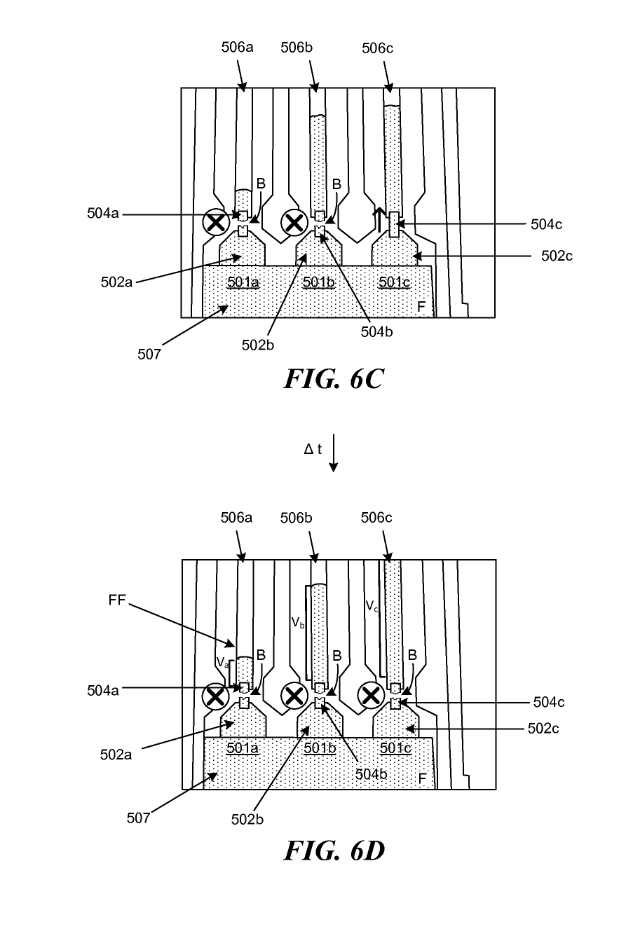

FIGS. 6A-6D show a series of time-lapsed top views of another capillarity-based device or analyzer 500 utilizing volume-metering elements 504 configured in accordance with the present technology to automatically dispense different fluid volumes to multiple pathways for downstream processing in a network from a single, user-filled source 507. As shown in FIG. 6A, for example, the device 500 can include three pathways 501 (individually labeled at 501a-501c) and a single source 507 servicing all three pathways. In other embodiments, the device 500 can include any number of pathways (e.g., two, four, etc.) and/or a separate source for each pathway and/or subset of pathways. Each of the pathways 501 can have different first materials 502 (individually labeled 502a-502c) so that one or more of the pathways 501a-501c can deliver the same or a different volume to the second materials 506 (individually labeled 506a-506c). For example, first material 502a can comprise nitrocellulose, first material 502b can comprise glass fiber, and first material 502c can comprise treated polyester. In other embodiments, any of the pathways can have the same and/or different pathway parameters to tailor the passable volume to the particular needs of the assay.

As shown in FIG. 6A, a fluid F can be added to the single source 507 (e.g., using a plastic transfer pipette). The device 500 can then be folded on itself, thereby connecting the first materials 502 to the second materials 506 via the respective volume-metering elements 504 (individually labeled 504a-504c) for each pathway (shown mid-folding in FIG. 6B). As shown in FIG. 6C, the volume metering elements 504a, 504b attached to the nitrocellulose first material 502a and the glass fiber first material 502b, respectively, can disconnect or shut-off before the volume-metering element 504c associated with the polyester first material 502c. As shown in FIG. 6D, the different final volumes delivered to the three pathways (Vₐ, V_{b}, V_{c}) can be observed in the different volumes occupied by the fluid front FF in the respective second materials 506a, 506b, 506c. In one particular embodiment, for example, the three fluid volumes delivered for the described pathway configurations of FIGS. 6A-6D can be about 9.5 μL, about 22 μL, and about 31 μL for pathway 505a, 505b and 505c, respectively. In other embodiments, however, different volumes of fluid may be delivered via the different pathways.

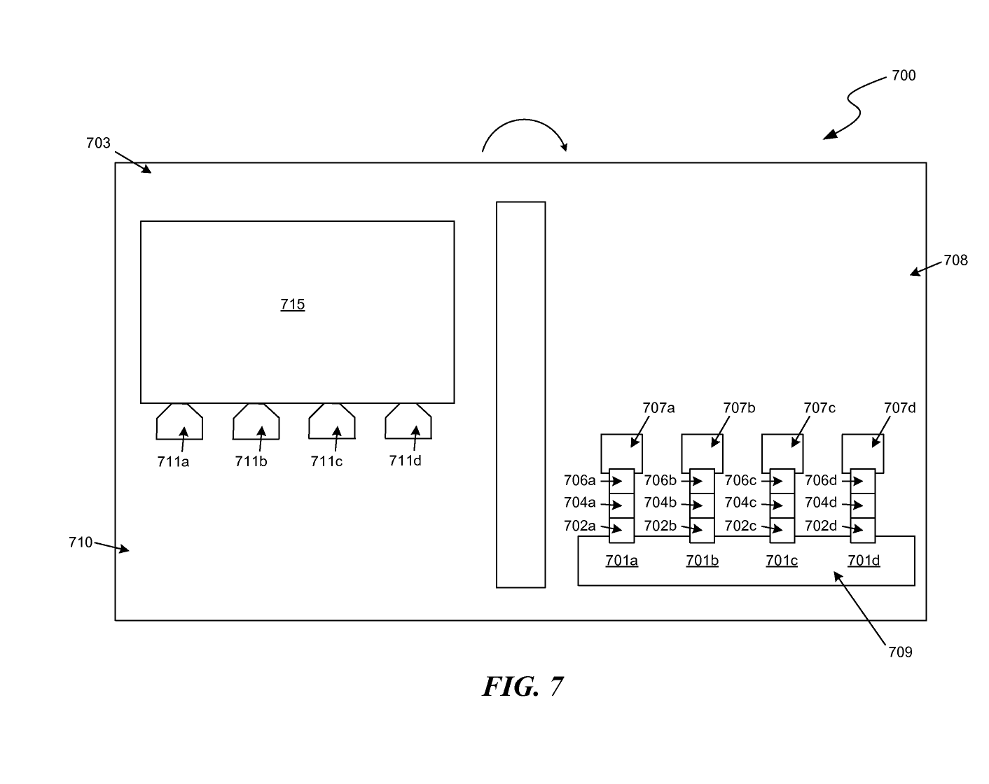

FIG. 7 is a top view of a microfluidic device or analyzer 700 configured in accordance with another embodiment of the present technology to automatically dispense different fluid volumes to multiple pathways for downstream processing in a network 715 from a single, user-filled well 709. As shown in FIG. 7, for example, a bottom layer 708 of the device 700 can include four pathways 701 (individually labeled at 701a-701d) and a single well 709 servicing all four pathways. In other embodiments, the device 700 can include any number of pathways (e.g., two, three, five, etc.) and/or a separate well for each pathway and/or subset of pathways. The first materials, second materials, volume-metering elements, and sources may be generally similar to the first materials, second materials, volume-metering elements, and sources described above, or they may have a different composition and/or configuration.

As shown in FIG. 7, each of the pathways 701 can have different first materials 702 (individually labeled 702a-702d) and/or volume-metering element 704 compositions (individually labeled 704a-704d) so that one or more of the pathways 701a-701d can deliver the same or a different volume to the respective second materials 706 (individually labeled 706a-706d). Each of the pathways 701a-701d can optionally include a source 707 (individually labeled 707a-707d) adjacent to and in fluid connection with the second materials 706a-706d. A top layer 710 of the device 700 can include one or more inlets 711 (individually labeled 711a-711d) adjacent to and in fluid connection with a network 715 of pathways (shown schematically in FIG. 7). The inlets 711a-711d can be positioned on the top layer 710 such that when the top layer 710 is folded onto the bottom layer 708 (or vice versa), the sources 707a-707d align with the respective inlets 711a-711d. As a result, once the housing 703 is folded and the sources 707a-707d make contact with the inlets 711a-711d, fluid from the sources 707a-707d can flow onto and through the inlets 711a-711d and to the network 715.

In operation, when fluid is added to the first materials 702a-702d (e.g., either directly or via the well 709), the fluid wicks (e.g., by capillarity force) from the first materials 702a-702d to the respective volume-metering elements 704a-704d to the respective second materials 706a-706d to the respective sources 707a-707d. Depending on the prescribed passable-volume for each pathway 701a-701d, the time it takes the passable volume to reach the source (and the respective volume-metering element 704 to dissolve and break) can be the same and/or different for all or a subset of the pathways 701a-701d.

In some embodiments, any of the pathways disclosed herein can include additional first and/or second materials in series along the same pathway connected by an additional flow-metering element (not shown). Further, in particular embodiments, a single pathway can have multiple branches (not shown) that converge and/or diverge. Examples of these and other suitable pathways and/or capillarity devices are described in PCT Application No. PCT/US2010/061675, filed Dec. 21, 2010, titled “CAPILLARITY-BASED DEVICES FOR PERFORMING CHEMICAL PROCESSES AND ASSOCIATED SYSTEMS AND METHODS,” and PCT. Application No. PCT/US2012/044060, filed Jun. 6, 2012, titled “REAGENT PATTERNING IN CAPILLARITY-BASED ANALYZERS AND ASSOCIATED SYSTEMS AND METHODS,” both of which are incorporated herein by reference in their entireties.

The capillarity-based devices and analyzers disclosed herein offer several advantages over conventional systems. First, conventional paper network assays require multiple fluid loading steps of specific volumes of fluid. In contrast, the present technology provides a multi-step chemical process with a single activation step. Also, the exact volume of fluid need not be added by the user to the source since the volume-metering element automatically dispenses the desired volume, regardless of the volume of fluid deposited into the source. Moreover, various methods of the present technology do not require a user to position the device in a specific orientation for operation.

Generally, devices configured in accordance with the present technology are expected to adapt the features of microfluidic devices to a porous wick (or paper) system, but without the need for external pumps, mechanical or electroosmotic, and without the need for pressure or vacuum sources to regulate the flow of fluid. Thus, no external force is necessary for the device to modulate the flow of fluid by means other than the capillary action (surface tension) of the wick and the associated absorbent pads.

In addition to the application of simple reagent loading, the present technology can be used in alternate contexts for controlling fluid volumes in paper networks. Specifically, these turn-off valves can be used further downstream in the paper network to meter volumes of reagents for interactions such as chemical dilution or reaction. Though the present technology demonstrates a range of volumes metered from about 10 μL to about 80 μL, one having skill in the art would understand how to extend this range by implementation of the volume-metering element in alternate materials and/or geometries.

The devices disclosed herein are also expected to improve the detection limits for analytes, such as simultaneous detection of two antigens from malarial parasites in blood, but at a manufacturing cost equal to that of conventional rapid diagnostic tests (RDTs). Further, results of a chemical process performed on the device can be read by eye or by cameras of mobile devices. For example, by capturing device detection spot intensities with mobile device cameras, blood antigen concentrations can be rapidly measured locally or remotely. This feature, for example, is expected to greatly aid in screening for the degree of subclinical infections at remote sites. This new approach to point-of-care diagnostics combines the sophistication of chemical processing developed in microfluidics with the simplicity and low cost of lateral flow immunoassays.

From the foregoing it will be appreciated that, although specific embodiments of the technology have been described herein for purposes of illustration, various modifications may be made without deviating from the spirit and scope of the technology. For example, the presence/configuration of the base or housing, the number of pathways, flow-metering elements, volume-metering features, the use of pre-wetted pads, the specific types of fluids, and the material choices for various components of the devices described above with reference to FIGS. 1A-6D may vary in different embodiments of the technology. Further, certain aspects of the new technology described in the context of particular embodiments may be combined or eliminated in other embodiments. For example, in the embodiments illustrated above, various combinations of flow-metering and volume-metering elements or features may be combined into a single device. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A device for performing chemical processes, the device comprising:
a source configured to receive one or more fluids;
a material; and
a dissolvable volume-metering element positioned between the source and the material and configured to provide a fluid connection between the source and the material,
wherein the volume-metering element is configured to at least partially dissolve and break the fluid connection between the source and material once a predetermined volume of fluid flows therethrough.

2. The device of claim 1 wherein the material is a first material and further comprising a second material adjacent to and in fluid connection with the source and the dissolvable volume-metering element.

3. The device of claim 1 wherein the volume-metering element comprises a cellulose material and is configured to deliver a fluid volume between about 1 μL and about 500 μL before breaking the fluid connection between the first material and the second material.

4. The device of claim 1 wherein:
the first material is composed of a first porous material selected from the group consisting of cellulose, a glass fiber material, paper, a polyester material, a nitrocellulose material, cellulose acetate, cellulose esters, polysulfone, polyether sulfone, polyacrilonitrile, polyamide, polyimide, polyethylene and polypropylene, polytetrafluoroethylene, polyvinylidene fluoride, and polyvinylchloride; and
the second material is composed of a second porous material selected from the group consisting of cellulose, a glass fiber material, paper, a polyester material, a nitrocellulose material, cellulose acetate, cellulose esters, polysulfone, polyether sulfone, polyacrilonitrile, polyamide, polyimide, polyethylene and polypropylene, polytetrafluoroethylene, polyvinylidene fluoride, and polyvinylchloride.

5. The device of claim 1 wherein the source is composed of a source material selected at least one of cellulose, a glass fiber material, paper, a polyester material, a nitrocellulose material, cellulose acetate, cellulose esters, polysulfone, polyether sulfone, polyacrilonitrile, polyamide, polyimide, polyethylene and polypropylene, polytetrafluoroethylene, polyvinylidene fluoride, polyvinylchloride, and a fluid in a container.

6. The device of claim 1, further comprising a support structure, wherein:
the volume-metering element further comprises a first portion in contact with the first material, a second portion in contact with the second material, and a third portion between the first portion and the second portion.
the first material and the second material are in contact with the support structure and the third portion of the volume-metering element is separated from the support structure by a gap.

7. The device of claim 1 wherein the volume-metering element is composed of a volume-metering element material selected from the group consisting of mannose, dextrose, fructose, galactose, trehalose, sucrose, lactose, maltose, mannitol, xylitol, sorbitol, polysaccharides, dextrans, maltodextrin, starch, and dextran derivatives.

8. A device for performing chemical processes, the device comprising:
a source configured to contain one or more fluids;
a first pathway adjacent to and in fluid connection with the source, wherein the first pathway includes—
a first feeder material adjacent to the source;
a first delivery material;
a first volume-metering element between the first feeder material and the first delivery material and configured to automatically and independently control or modify a first volume of fluid flow between the first feeder material and the first delivery material;
a second pathway adjacent to and in fluid connection with the source, wherein the second pathway includes—
a second feeder material adjacent to the source;
a second delivery material;
a second volume-metering element between the second feeder material and the second delivery material and configured to automatically and independently control or modify a second volume of fluid flow between the second feeder material and the second delivery material.

9. The device of claim 8 wherein the first delivery material and the second delivery material are in fluid communication.

10. The device of claim 8 wherein the first volume-metering element is configured to deliver a first volume of fluid to the first pathway and the second volume-metering element is configured to deliver a second volume of fluid to the second pathway that is different than the first volume.

11. The device of claim 8 wherein the first feeder material is different than the second feeder material.

12. The device of claim 8 wherein the first volume-metering element has a different cross-sectional area than that of the second volume-metering element.

13. The device of claim 8 wherein the first volume-metering element is made of a first material and the second volume-metering element is made of a second material that is different than the first material.

14. The device of claim 8 wherein:
the first feeder material is different than the second feeder material; and
the first volume-metering element has a different cross-sectional area than that of the second volume-metering element.

15. The device of claim 8 wherein:
the first feeder material is different than the second feeder material; and
the first volume-metering element is made of a first material and the second volume-metering element is made of a second material that is different than the first material.

16. A device for performing chemical processes, the device comprising:
a foldable housing having a first layer and a second layer, wherein the housing is moveable between an open position and a closed position;
a source positioned on the first layer of the housing and configured to receive one or more fluids;
a first material positioned on the first layer of the housing adjacent to and in fluid connection with the source;
a second material positioned on the first layer of the housing; and
a dissolvable volume-metering element positioned on the second layer of the housing such that when the housing is in the closed position, the volume-metering element is positioned between the first material and the second material so as to provide a fluid connection between the first material and the second material, and wherein the volume-metering element is configured to at least partially dissolve such that the fluid connection between the first and second materials is broken once a predetermined volume of fluid flows therethrough.

17. A capillarity-based method for analyzing a fluid, the method comprising:
depositing a first volume of fluid at a fluid source, wherein the fluid source is adjacent to a feeder material;
delivering a second volume of fluid from the feeder material to a delivery material via a dissolvable volume-metering element;
wherein the first and second volumes are different.

18. The method of claim 17, further comprising:
dissolving the volume-metering element so that the feeder material and the delivery material are no longer in fluid connection.

19. The method of claim 17 wherein the fluid source is adjacent to a first feeder material and a second feeder material, and wherein the method further comprises:
delivering a third volume of fluid from the second feeder material to a second delivery material via a second dissolvable volume-metering element;
wherein the first, second and third volumes are different.

20. The method of claim 17, further comprising:
dissolving the first volume-metering element so that the first feeder material and the first delivery material are no longer in fluid connection; and
dissolving the second volume-metering element so that the second feeder material and the second delivery material are no longer in fluid connection.

21. The method of claim 20, wherein dissolving the first volume-metering element occurs before dissolving the second volume-metering element.
